(19) United States
(12) Patent Application Publication
(10) Pub. No.: US 2022/0151565 A1
(43) Pub. Date: May 19, 2022

(54) SIGNAL QUALITY INDEX EVALUATION CIRCUIT

(71) Applicants: Inventec (Pudong) Technology Corporation, Shanghai (CN); INVENTEC CORPORATION, Taipei (TW)

(72) Inventors: JONATHAN HANS SOESENO, Taipei (TW); Trista Pei-Chun Chen, Taipei (TW); Jiun-Han Chen, Taipei (TW); Davide Burba, Taipei (TW)

(21) Appl. No.: 17/157,037

(22) Filed: Jan. 25, 2021

(30) Foreign Application Priority Data
Nov. 19, 2020 (CN) 202011297872.0

(51) Int. Cl. A61B 5/00; G16H 50/30; G16H 50/70

(52) U.S. Cl. CPC A61B 5/7221; A61B 5/725; A61B 5/7225; A61B 5/02416; G16H 50/30; G16H 50/70; A61B 5/7246

(57) ABSTRACT

A signal quality index evaluation circuit, comprises: a surrounding sensor; a zero-phase filter; and an evaluation circuit. The surrounding sensor senses its surrounding to generate a reference correction signal. The zero-phase filter is configured to generate a clean biological signal according to a biological signal and the reference correction signal, wherein the clean biological signal includes a plurality of period signals, and each one of the period signals has a biological value. The evaluation circuit is configured to calculate norm range according to the clean biological signal and one or more of the biological values of the period signals, and determine a difference between each one of the biological values corresponding to each one of the period signals and the norm range, the evaluation circuit is further configured to calculate and output a signal quality index according to the differences.

### CROSS-REFERENCE TO RELATED APPLICATIONS

This non-provisional application claims priority under 35 U.S.C. § 119(a) on Patent Application No(s). 202011297872.0 filed in China on Nov. 19, 2020, the entire contents of which are hereby incorporated by reference.

### BACKGROUND

### 1. Technical Field

This disclosure relates to a signal quality index evaluation circuit, especially to a signal quality index evaluation circuit that is able to exclude noise existed in a biological signal and evaluate the quality of the biological signal.

### 2. Related Art

Cardiovascular disease has been confirmed to have high association with heart rate and blood pressure. Blood pressure means the pressure generated by the blood in the circulating system. Since blood pressure is closely related to the force, frequency of heart beat, as well as the elasticity and diameter of the arterial wall, blood pressure is often measured for the purposes of diagnosis and treatment. If blood pressure is not properly controlled, it may further lead to problems such as heart disease, stroke or heart failure. Therefore, it is even more important to improve the accuracy of blood pressure measurement by using artificial intelligence technology.

The technology of blood pressure measurement has been developed for many years, and the method of measuring blood pressure can be divided into cuff measurement and cuff-less measurement based on the method of calculating blood pressure. Comparing to cuff measurement, cuff-less measurement is easier to operate and can perform continuous measurement.

Cuff-less measurement in recent years comprises electrocardiogram (ECG) and photoplethysmography (PPG) measurements. However, the signals obtained through these two types of measurements are usually not ideal due to base line wander, power line interference (PLI) and myoelectric noise. Even though the signal is processed with cut-off frequency, it is still difficult for medical staff or researchers to determine whether the processed signal is accurate merely by viewing it.

### SUMMARY

Accordingly, this disclosure provides a signal quality index evaluation circuit to meet the above needs.

According to one or more embodiment of this disclosure, a signal quality index evaluation circuit, comprising: a surrounding sensor, configured to sense surrounding to generate a reference correction signal; a zero-phase filter, in communication connection with the surrounding sensor, the zero-phase filter is configured to generate a clean biological signal according to a biological signal and the reference correction signal, wherein the clean biological signal includes a plurality of period signals, and each one of the period signals has a biological value; and an evaluation circuit, in communication connection with the zero-phase filter, the evaluation circuit is configured to calculate norm range according to the clean biological signal and one or more of the biological values of the period signals, and determine a difference between each one of the biological values corresponding to each one of the period signals and the norm range, the evaluation circuit is further configured to calculate and output a signal quality index according to the differences.

### BRIEF DESCRIPTION OF THE DRAWINGS

The present disclosure will become more fully understood from the detailed description given hereinbelow and the accompanying drawings which are given by way of illustration only and thus are not limitative of the present disclosure and wherein:

FIG. 1 is a block diagram of a signal quality index evaluation circuit according to an embodiment of the present disclosure;

FIG. 2A is an exemplary diagram illustrating the waveform of ECG signal;

FIG. 2B is a waveform of a clean biological signal corresponding to the ECG signal shown in FIG. 2A;

FIG. 2C is an average waveform corresponding to the clean biological signal shown in FIG. 2B;

FIG. 3A is an exemplary diagram illustrating the distribution of biological values according to an embodiment of the present disclosure;

FIG. 3B is another exemplary diagram illustrating the distribution of biological values according to an embodiment of the present disclosure;

FIG. 4A is an exemplary waveform illustrating a pulse signal;

FIG. 4B is a waveform of the clean biological signal corresponding to the pulse signal of FIG. 4A;

FIG. 5A is a statistical chart according to an estimated signal quality index corresponding to 500 clean biological signals within 30 seconds; and

FIG. 5B is a statistical chart according to an estimated signal quality index corresponding to 500 clean biological signals within 30 seconds.

### DETAILED DESCRIPTION

It should be first noted that, the signal quality index evaluation circuit according to the present disclosure can generate reference signal by sensing surroundings. Therefore, when sensing a biological signal, such as ECG or PPG, the biological signal can be processed according to the reference signal, to output a more accurate biological signal.

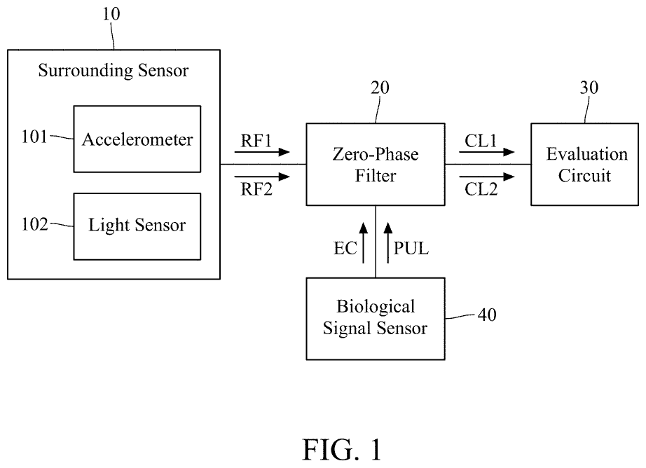

Please refer to FIG. 1, FIG. 1 is a block diagram of a signal quality index evaluation circuit according to an embodiment of the present disclosure. The signal quality index evaluation circuit according to an embodiment of the present disclosure comprises a surrounding sensor 10, a zero-phase filter 20 and an evaluation circuit 30, wherein the surrounding sensor 10 is in communication connection with the zero-phase filter 20; the zero-phase filter 20 is in communication connection with the evaluation circuit 30.

The surrounding sensor 10 is configured to sense its surrounding to generate a reference correction signal RF1 or RF2. That is, the surrounding sensor 10 is configured to sense a movement of a subject or a light intensity of its surrounding. The zero-phase filter 20 is configured to generate a clean biological signal CL1 or CL2 according to a biological signal and the reference correction signal RF1 or RF2, wherein the clean biological signal CL1 or CL2 includes a plurality of period signals, and each one of the period signals has a biological value. Accordingly, the evaluation circuit 30 can calculate a norm range according to the biological values of the period signals of the clean biological signal CL1 or CL2, and further calculate a difference between the biological value of each one of the period signals and the norm range, to calculate and output a signal quality index (SQI) according to the differences.

In short, the biological signal can be an electro-cardio signal EC or a pulse signal PUL. Therefore, the surrounding sensor 10 preferably includes an accelerometer 101 and a light sensor 102. The zero-phase filter 20 can process the electro-cardio signal EC or the pulse signal PUL obtained by a biological signal sensor 40 according to the signals obtained by the accelerometer 101 and the light sensor 102 to generate the corresponding clean biological signal CL1 or CL2. Therefore, the evaluation circuit 30 can calculate the signal quality index of the clean biological signal CL1 or CL2, to determine if the clean biological signal CL1 or CL2 is a usable signal. In the following, the biological signal will be described as the electro-cardio signal EC.

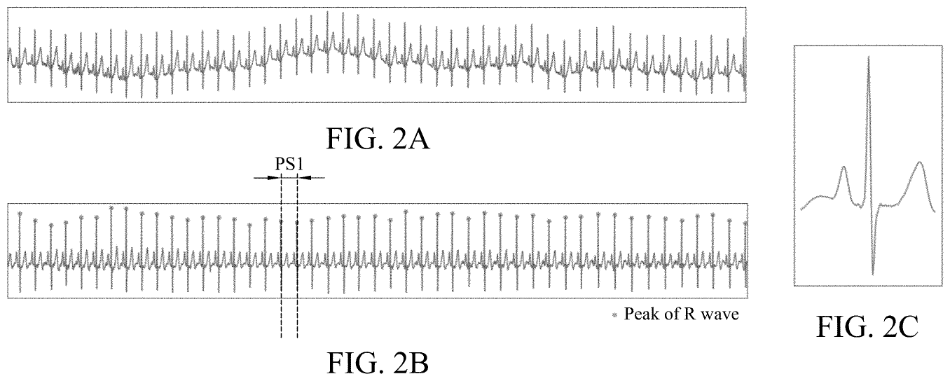

Please refer to both FIG. 1 and FIG. 2A, wherein FIG. 2A is an exemplary diagram illustrating the waveform of ECG signal. Since the electro-cardio signal EC obtained by the biological signal sensor 40 is raw data, the waveform shown FIG. 2A may have baseline wander due to movement or movement caused by breathing or walking of the subject.

Therefore, the accelerometer 101 can sense the noise introduced due to the movement of the subject at the same time when the biological signal sensor 40 senses the electro-cardio signal EC. For example, if the subject is walking while the biological signal sensor 40 is sensing the electro-cardio signal EC, a 2 Hz noise may get introduced into the sensed electro-cardio signal EC. Therefore, the accelerometer 101 can be used to sense the movement signal generated due to the movement of the subject, and the movement signal can be used as the reference correction signal. In other words, the reference correction signal RF1 is the signal corresponding to the 2 Hz noise.

When the zero-phase filter 20 receives the electro-cardio signal EC and the reference correction signal RF1, the zero-phase filter 20 calculates the frequency of the noise generated by the movement of the subject (for example, the 2 Hz noise may get introduced due to walking) according to the reference correction signal RF1, and filters out a signal with, for example, 3 Hz-45 Hz frequency or 0.5 Hz-50 Hz by passband filtering and stopband filtering, and uses the filtered signal as the clean biological signal CL1.

Specifically, although a convention frequency filter can filter out noise signal, it may cause the increase of phase shift or other wanted phenomenon at the same time. Therefore, with the zero-phase filter 20, it is able to avoid introducing phase shift during the process of filtering, and to filter out signal with wanted frequency.

The waveform of the clean biological signal CL1 is shown as FIG. 2B, FIG. 2B is a waveform of the clean biological signal CL1 corresponding to the electro-cardio signal EC shown in FIG. 2A, and the clean biological signal CL1 has a plurality of period signals PS1, one period signal PS1 is, for example, a signal from one peak (the peak of the R wave) to the next peak, or from one wave trough to the next wave trough, the present disclosure does not limit the way of dividing the period signal PS1.

The evaluation circuit 30 then performs signal usability analysis on one or more period signals PS1 of the clean biological signal CL1, wherein the analysis can include average analysis, correlation analysis or distribution model analysis.

The evaluation circuit 30 performing average analysis on the clean biological signal CL1 can be first calculating a norm range based on one or more period signals PS1, and calculating the norm range can be calculating one or more averages of the biological values, wherein one period signal PS1 obtained by the average can be as shown in FIG. 2C. However, the method of calculating the norm range can also be calculating a median or a mode of the biological values, the present disclosure is not limited thereto. Calculating the average of the biological values can be implemented with the following equation (1):

$$\overline{c} = \frac{{Σ}_{i = 1}^{N} {c}_{i} ( x )}{N} - ( 1 ) \qquad$$

wherein c̅ is the average of the biological values of the one or more period signals PS1 of the clean biological signal CL1; cᵢ(x) is the biological value of each one of the period signals PS1 of the clean biological signal CL1; N is the total number of the period signals PS1.

The evaluation circuit 30 calculates the signal quality index based on the differences between each one of the biological values and the average. That is, the evaluation circuit 30 can calculate the number of the differences that fall within a range of a threshold value, and use the ratio of the number of the differences that fall within the range of the threshold value to the total number of the differences as the signal quality index.

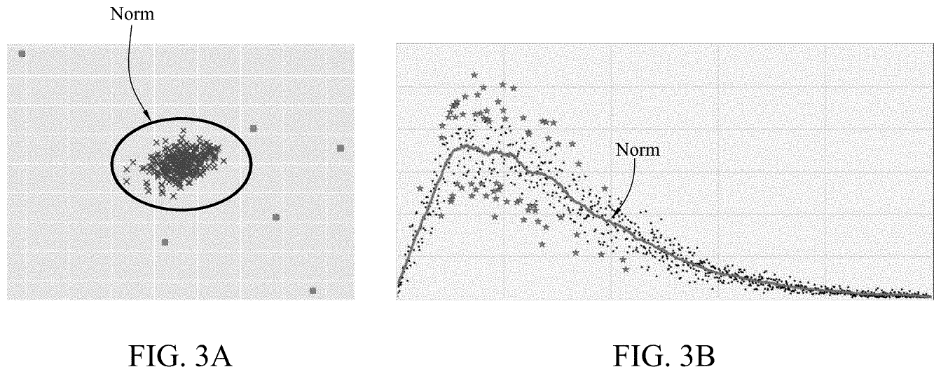

Further, please refer to FIG. 3A, wherein FIG. 3A is an exemplary diagram illustrating the distribution of biological values according to an embodiment of the present disclosure. Each point shown in FIG. 3A represents a high-dimensional biological value of one period signal.

After the evaluation circuit 30 calculates the average c̅, the average c̅ can be uses as a center point for defining the norm range Norm. Each biological value has a distance from the center point, which is the average c̅. The evaluation circuit 30 can determine if the distance corresponding to each biological value is not bigger than the distance between the center point of the norm range Norm to circumference of its ellipse, and determine the biological value with the distance that is not bigger than the distance between the center point of the norm range Norm to its circumference a usable period signal PS1. The evaluation circuit 30 further calculates the ratio of the number of the biological values with the distance that is not bigger than the distance between the center point of the norm range Norm to its circumference to the total number of the biological values, and uses the ratio as the signal quality index. In short, the biological value that falls within the norm range Norm is the usable period signal PS1 of the clean biological signal CL1; the biological value that falls outside the norm range Norm may be abnormal period signal PS1 (shown as a square in FIG. 3A) of the clean biological signal CL1. Specifically, since the biological values of one subject mostly fall within a range, the evaluation circuit 30 can calculate the ratio of the biological values that fall within the norm range Norm to the total number of the biological values, and use the calculated ratio as the signal quality index. Further, since one dimension may correspond to one tolerance (for example, standard deviation), that is, each dimension may correspond to a separate norm range, so as shown in FIG. 3A, the norm range Norm is presented as an ellipsoid, and the biological values surrounded by it are the biological values within the tolerance (standard deviation).

When the number of the biological values that fall within the norm range Norm reaches a threshold ratio, it means the signal quality index of the clean biological signal CL1 is high, that is, the clean biological signal CL1 is suitable for subsequent calculation of blood pressure. On the contrary, when the number of the biological values that fall within the norm range Norm does not reach the threshold ratio, it means the signal quality index of the clean biological signal CL1 is low, and that the clean biological signal CL1 is not suitable for subsequent calculation of blood pressure.

Please refer to FIG. 3B, FIG. 3B is another exemplary diagram illustrating the distribution of biological values according to an embodiment of the present disclosure. Each point shown in FIG. 3B represents a high-dimensional biological value of one period signal. Similar to FIG. 3A, the norm range Norm can be the curve shown in FIG. 3B, and the data point presented as a dot is the period signal PS1 with the distance between the clean biological signal CL1 and the norm range Norm is within a preset range; the data point presented as a star is the period signal PS1 with the distance between the clean biological signal CL1 and the norm range Norm exceeds the preset range. Therefore, the evaluation circuit 30 can calculate the signal quality index of the clean biological signal CL1 based on the ratio of the dot data points to the total number of the data points shown in FIG. 3B.

As described above, the evaluation circuit 30 can also analyze the clean biological signal CL1 by a distribution model. That is, a Gauss distribution model can be established the biological values of the period signals PS1 of the clean biological signal CL1, wherein the confidence interval of the Gauss distribution model is the norm range, and the average c̅ is the peak value of the Gauss distribution model. The evaluation circuit 30 calculates the differences between the biological values and the average c̅, and uses the ratio of the number of the differences that fall within the range of the threshold value to the total number of the differences as the signal quality index. The evaluation circuit 30 determines if the ratio reaches the ratio of the confidence interval, and determines the clean biological signal CL1 is suitable for the subsequent calculation of the blood pressure when the number of the differences that fall within the range of the threshold value reaches the ratio of the confidence interval.

Further, the evaluation circuit 30 analyzes the clean biological signal CL1 by correlation can be the evaluation circuit 30 calculating the correlation between each biological value of the clean biological signal CL1 and the average, and calculating the signal quality index of the clean biological signal CL1 based on the calculated correlation. Calculating the signal quality index of the clean biological signal CL1 can be implemented as the following equation (2):

$${C}_{SQI} ( x ) = \frac{{Σ}_{i = 1}^{N} Corr ( {c}_{i} ( x ) , \overline{c} ( x ) )}{N} - ( 2 ) \qquad$$

wherein, C_{SQI}(x) is the signal quality index; Corr(cᵢ(x), c̅(x)) is the correlation between the biological values cᵢ (x) of each one of the period signals PS1 and the average c̅(x) of the period signals; N is the total number of the period signals PS1. Therefore, when the signal quality index C_{SQI}(x) reaches an expected value, it means the clean biological signal CL1 is suitable for the subsequent calculation of the blood pressure.

Take FIGS. 2A and 2B as example, when the electro-cardio signal EC is a raw data as shown in FIG. 2A, its signal quality index C_{SQI}(x) is only 66%; when the electro-cardio signal EC is the clean biological signal CL1 as shown in FIG. 2B, its signal quality index C_{SQI}(x) can be up to 97%. According to one or more embodiment described above, not only the electro-cardio signal EC can be processed to obtain the clean biological signal CL1, the clean biological signal CL1 can be further determined if it's a usable signal, so that the subsequent calculation of the blood pressure can be more accurate.

Similar to the signal processing and methods of calculating the signal quality index of the electro-cardio signal EC, the pulse signal PUL can also be processed by the zero-phase filter 20 to generate the corresponding clean biological signal CL2. The evaluation circuit 30 then can calculate the signal quality index of the clean biological signal CL2.

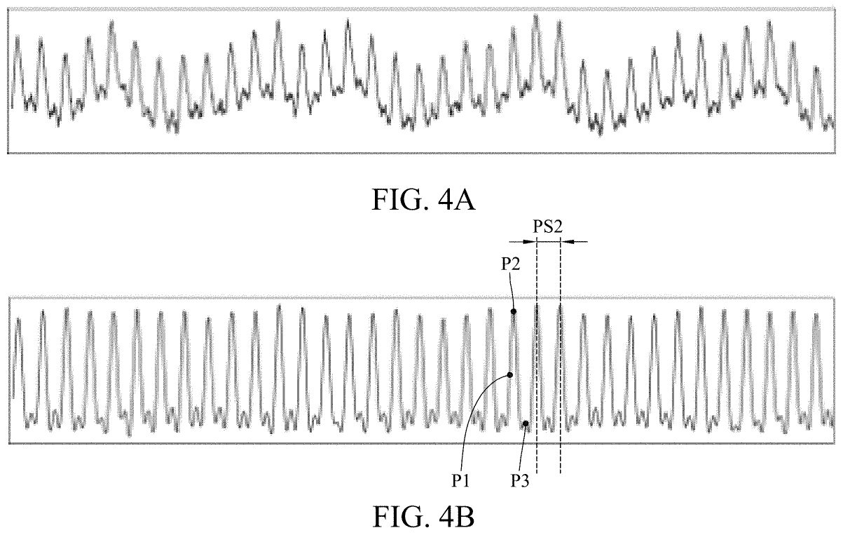

Specifically, light sensor 102 can sense its surrounding to sense the noise introduced due to surrounding light at the same time when the biological signal sensor 40 senses the pulse signal PUL. For example, if there is surrounding light when the biological signal sensor 40 is sensing the pulse signal PUL, the sensed pulse signal PUL may be introduced with the noise caused by the surrounding light, as shown in the waveform of the 30 seconds pulse signal in FIG. 4A. Therefore, the light sensor 102 can be used to sense the surrounding light to generate light signal, and uses the light signal as the reference correction signal RF2.

When the zero-phase filter 20 receives the pulse signal PUL and the reference correction signal RF2 (the light signal sensed by the light sensor 102), the zero-phase filter 20 calculates the frequency of the noise generated by the surrounding light according to the reference correction signal RF2, and filters out a signal with, for example, 0.5 Hz-50 Hz frequency by passband filtering, and uses the filtered signal as the clean biological signal CL2.

The waveform of the clean biological signal CL2 is shown as FIG. 4B, the evaluation circuit 30 then performs signal usability analysis on one or more period signals PS2 of the clean biological signal CL2, wherein the analysis not only can include average analysis, correlation analysis or distribution model analysis described above, but also moving average analysis.

Specifically, as shown in FIG. 4B, each point P1˜P3 on the 30 seconds waveform corresponds to a normalized value, and the evaluation circuit 30 establishes a histogram based on the number of the normalized values of the points P1˜P3, wherein the horizontal axis is the normalized value, and the vertical axis is the number of the normalized values. The evaluation circuit 30 can calculate an estimated signal quality index based on the skewness of the histogram. Calculating the estimated signal quality index based on the skewness of the histogram can be implemented as the following equation (3):

$${S}_{SQI} = \frac{{{Σ}_{i = 1}^{N} [ {x}_{i} - \overline{x} / σ ]}^{3}}{N} - ( 3 ) \qquad$$

wherein, xᵢSMAS_OUT_{SQI}(x)C_{SQI} (x) is the biological value of each one of the period signals P2 of the clean biological signal CL2; σ is a standard deviation of the biological value of each one of the period signals P2; N is a signal length of the clean biological signal CL2. By equation (3), the estimated signal quality index (S_{SQI}) of the 30 seconds pulse signal PUL shown in FIG. 4A is 0.5610; the estimated signal quality index (S_{SQI}) of the 30 seconds clean biological signal CL2 shown in FIG. 4B is 0.7499.

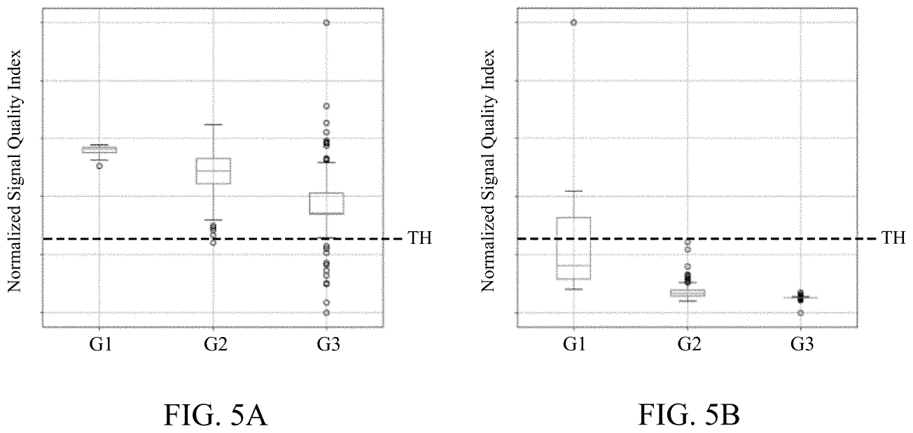

However, as shown in FIG. 5A, which is a statistical chart according to an estimated signal quality index corresponding to 500 clean biological signals CL2 within 30 seconds If the estimated signal quality index is only calculated based on the skewness of the histogram, the clean biological signal with low estimated signal quality index may be mistakenly considered as a signal with high estimated signal quality index. In other words, the three group of period signals G1˜G3 respectively represents the “good”, “ordinary” and “bad” period signals PL2 of the clean biological signals CL2. Since the second group of period signal G2 and the third group of period signal G3 distribute at a wider range, the three groups of period signals G1˜G3 overlap with each other to a large extent on the vertical axis. That is, the “bad” third group of period signal G3 also covers high signal quality index. Therefore, even if a signal quality threshold TH is set, it is still possible to obtain the “bad” third group of period signal G3.

Hence, the present disclosure first calculates the estimated signal quality index (S_{SQI}) associated with the skewness, and calculates the signal quality index (SQI of SMAS_OUT) based on the estimated signal quality index (S_{SQI}) and the moving average. Calculating the signal quality index of the clean biological signals CL2 within a sampling period based on the moving average can be implemented by the following equation (4):

$${SMAS\_OUT}_{SQI} ( x ) = {( \frac{1}{{σ}_{M A} ( x )} )}^{2} \times {S}_{SQI} ( x ) \times ( 1 - Flat ( x ) ) \times \frac{1}{1 + n} - ( 4 ) \qquad$$

wherein, σ_{MA}(x) is a standard deviation of the moving average of the clean biological signal CL2 within the sampling period, the sampling period in this embodiment is 30 seconds; c̅(x) is a function of the waveform of the clean biological signal CL2, is 0 when the signal is a non-flat signal, and is 1 when the signal is a flat signal; c̅(x) is the number of the peak values of the period signals PS2 that are outliers, take FIG. 5A as an example, is the amount of the peak values that are greater than Q3+(Q3−Q1)×1.5.

Please then refer to FIG. 5B, FIG. 5B is a statistical chart according to an estimated signal quality index corresponding to 500 clean biological signals CL2 within 30 seconds. That is, FIG. 5B is the statistical chart established with the signal quality index (SQI of SMAS_OUT) calculated using equation (4). Since equation (4) further takes the standard deviation σ_{MA}(x) of the moving average into consideration, as shown in the statistical chart presented in FIG. 5B, the three groups of period signals G1˜G3 have little overlap on the vertical axis, and the “bad” third group of period signal G3 distributes narrower than in FIG. 5A. Thus, it is able to distinguish the difference of the three groups of period signals G1˜G3.

Accordingly, not only can the distribution range and the extent of overlap on the vertical axis of the second group of period signals G2 and the third group of period signals G3 be reduced, the clean biological signal CL2 with a higher signal quality index can further be obtained by adjusting the signal quality threshold TH.

It should also be noted that, the signal quality index corresponds to the electro-cardio signal EC is preferably calculated by the average analysis, the correlation analysis or the distribution model analysis; the signal quality index corresponds to the pulse signal PUL is preferably calculated by the moving average analysis. However, analysis of the signal quality index of the electro-cardio signal EC can also include the moving average analysis, and the analysis of the signal quality index of the pulse signal PUL can also include the average analysis, the correlation analysis and the distribution model analysis, the present disclosure is not limited thereto.

In view of the above description, the signal quality index evaluation circuit according to one or more embodiment of the present disclosure can efficiently exclude the noise in the elector-cardio signal caused by the movement or breathing of a subject, and can also efficiently exclude the noise in the pulse signal caused by the surrounding light, and avoid unwanted phase shift being introduced during the process of frequency filter. Further, during subsequent signal analysis, it is able to identify the biological signals suitable for the calculation of blood pressure.

The present disclosure has been disclosed above in the embodiments described above, however it is not intended to limit the present disclosure. It is within the scope of the present disclosure to be modified without deviating from the essence and scope of it. It is intended that the scope of the present disclosure is defined by the following claims and their equivalents.

What is claimed is:

1. A signal quality index evaluation circuit, comprising:
a surrounding sensor, configured to sense surrounding to generate a reference correction signal;
a zero-phase filter, in communication connection with the surrounding sensor, the zero-phase filter is configured to generate a clean biological signal according to a biological signal and the reference correction signal, wherein the clean biological signal includes a plurality of period signals, and each one of the period signals has a biological value; and
an evaluation circuit, in communication connection with the zero-phase filter, the evaluation circuit is configured to calculate norm range according to the clean biological signal and one or more of the biological values of the period signals, and determine a difference between each one of the biological values corresponding to each one of the period signals and the norm range, the evaluation circuit is further configured to calculate and output a signal quality index according to the differences.

2. The signal quality index evaluation circuit according to claim 1, wherein the evaluation circuit calculates the norm range comprises: the evaluation circuit calculates an average of one or more of the biological values, and uses the average as the norm range.

3. The signal quality index evaluation circuit according to claim 1, wherein the evaluation circuit calculates the norm range comprises: the evaluation circuit establishes a distribution model with one or more of the biological values, and uses a confidence interval of the distribution model as the norm range.

4. The signal quality index evaluation circuit according to claim 1, wherein the evaluation circuit calculates the signal quality index according to the differences is: the evaluation circuit calculates a ratio of the number of differences being smaller than a threshold value to the total number of the differences, and uses the ratio as the signal quality index.

5. The signal quality index evaluation circuit according to claim 1, wherein the evaluation circuit calculates the signal quality index according to the differences is: the evaluation circuit calculates a correlation according to each one of the differences, and calculates the signal quality index according to the correlation and the total number of the period signals.

6. The signal quality index evaluation circuit according to claim 5, wherein the evaluation circuit calculates the signal quality index according to the correlation and the total number of the period signals is: the evaluation circuit calculates the signal quality index according to a signal quality index equation, wherein the signal quality index equation is:
$${C}_{SQI} ( x ) = \frac{{Σ}_{i = 1}^{N} Corr ( {c}_{i} ( x ) , \overline{c} ( x ) )}{N} \qquad$$
wherein, C_{SQI} (x) is the signal quality index; cᵢ (x) is the biological value of each one of the period signals; c̅(x) is an average of each one of the period signals and the biological value for calculating the norm range; Corr(cᵢ(x), c̅(x)) is a correlation between the biological value of each one of the period signals and the average of the biological values; N is the total number of the period signals.

7. The signal quality index evaluation circuit according to claim 1, wherein the evaluation circuit calculates the signal quality index according to the differences is: the evaluation circuit calculates a skewness based on the differences, and calculates the signal quality index according to the skewness and a moving average.

8. The signal quality index evaluation circuit according to claim 7, wherein the evaluation circuit calculates the signal quality index according to the skewness and the moving average is: the evaluation circuit calculates the signal quality index according to a signal quality index equation, wherein the signal quality index equation is:
$${SMAS\_OUT}_{SQI} ( x ) = {( \frac{1}{{σ}_{M A} ( x )} )}^{2} \times {S}_{SQI} ( x ) \times ( 1 - Flat ( x ) ) \times \frac{1}{1 + n}$$
wherein, SMAS_OUT_{SQI}(x)C_{SQI}(x) is the signal quality index; σ_{MA}(x) is a standard deviation of the moving average of the clean biological signal corresponding to a sampling period; c̅(x) is an estimated signal quality index associated with the skewness; c̅(x) is a function of the waveform of the clean biological signal; c̅(x) is the number of the peak values of the period signals that are outliers.

9. The signal quality index evaluation circuit according to claim 8, wherein the evaluation circuit calculates the estimated signal quality index according to an estimated signal quality index equation, wherein the estimated signal quality index equation is:
$${S}_{SQI} = \frac{{{Σ}_{i = 1}^{N} [ {x}_{i} - \overline{x} / σ ]}^{3}}{N}$$
wherein, xᵢSMAS_OUT_{SQI}(x)C_{SQI}(x) is the biological value of each one of the period signals; x̅ is an average of the biological value of each one of the period signals for calculating the norm range; σ is a standard deviation of the biological value of each one of the period signals; N is a signal length of the clean biological signal.

10. The signal quality index evaluation circuit according to claim 1, wherein the surrounding sensor comprises an accelerometer and a light sensor.
